(19) United States
(12) Patent Application Publication
(10) Pub. No.: US 2003/0134817 A1
(43) Pub. Date: Jul. 17, 2003

(54) SIV derived lentiviral vector systems

(75) Inventor: Vicente Planelles, Salt Lake City, UT (US)

Correspondence Address: NIXON PEABODY LLP, ATTENTION: DAVID RESNICK, 101 FEDERAL STREET, BOSTON, MA, 02110, US

(73) Assignee: University of Rochester

(21) Appl. No.: 10/304,988

(22) Filed: Nov. 26, 2002

Related U.S. Application Data
(63) Continuation of application No. PCT/US01/17458, filed on May 30, 2001.
(60) Provisional application No. 60/207,916, filed on May 30, 2000.
(60) Provisional application No. 60/287,433, filed on Apr. 30, 2001.

(51) Int. Cl. A61K 48/00; C12N 15/867; C12N 5/08; C12N 7/00

(52) U.S. Cl. 514/44; 424/93.2; 435/235.1; 435/456; 435/366

(57) ABSTRACT

An SIV-derived vector system for transferring a nucleic acid sequence encoding a target molecule to a host cell is described. The vector system comprises a transfer vector containing the nucleic acid segment of interest, a packaging vector which is deleted for at least one of the accessory proteins (vif, vpr, vpx, and/or nef), and an env vector containing an envelope protein which is not an SIV envelope protein. In one embodiment the vector system is Rev-independent.

### FIELD OF THE INVENTION

The present invention is directed to a vector system wherein multiple SIV-derived lentiviral vectors are used to transfer nucleic acid segments to host cells.

### BACKGROUND OF THE INVENTION

In recent years considerable effort has been directed at gene delivery techniques for gene therapy. The aim of gene therapy is to modify the genetic material of living cells to achieve therapeutic benefit. Gene therapy involves the insertion of a functional gene into a cell, with the aim or replacing an absent or defective gene, or fighting an infectious agent or a tumor. Gene delivery methods include, for example, vectors such as viral vectors, liposomes, naked DNA, adjuvant-assisted DNA, gene gun, catheters, etc. The different techniques used depend in part upon the gene being transferred and the purpose therefore. Thus, for example, there are situations where only a short-term expression of the gene is desired in contrast to situations where a longer term, even permanent expression of the gene is desired.

A prime requirement for successful gene therapy is the sustained expression of the therapeutic gene without any adverse effect on the recipient. A highly desirable vector for delivering the therapeutic gene would be generated at high titers, integrate into target cells including non-dividing cells, and would have little or no associated immune reactions.

Vectors that have been looked at include both DNA viral vectors and RNA viral vectors. For example, DNA vectors include pox vectors such as orthopox or avipox vectors (see, e.g., U.S. Pat. No. 5,656,465), herpes virus vectors, such as herpes simplex I Virus (HSV) vector [Geller, A. I. et al., J. Neurochem. 64:487 (1995); Lim, F., et al., DNA Cloning: Mammalian Systems, D. Glover, Ed., Oxford Univ. Press, Oxford, England (1995); Geller, A. I. et al., Proc. Natl. Acad. Sci., U.S.A. 90:7603 (1993)]; Adenovirus vectors [Legal Lasalle et al., Sci. 259-988 (1993); Davidson et al., Nat. Genet. 3:219 (1993); Yang et al., J. Virol., 69:2004 (1995)]; and Adeno Associated Virus Vectors [Kaplitt, M. G., et al., Nat. Genet. 8;148 (1994)]. Retroviral vectors include Moloney murine leukemia viruses (MMLV) and human immunodeficiency viruses (HIV) [See, U.S. Pat. No. 5,665,577].

Concern with gene delivery systems has been expressed as a result of concerns such as antigenicity, viral recombination, efficiency, etc. A wide range of approaches to dealing with such concerns has been proposed. For example, further tailoring of vector constituents, and/or making such vectors self-inactivating. Moreover, the choice of certain classes of vectors can reduce such problems.

Retroviral vectors can be used to infect a host cell and have the genetic material integrated into that host cell with high efficiency. One example of such a vector is a modified Moloney murine leukemia virus (MMLV), which has had its packaging sequences deleted to prevent packaging of the entire retroviral genome. However, that retrovirus does not transduce resting cells. Additionally, since many retroviruses typically enter cells via specific receptors, if the specific receptors are not present on a cell or are not present in large enough numbers, the infection is either not possible or is inefficient. Concerns have also been expressed as a result of outbreaks of wild-type viruses from the recombinant MMLV producing cell lines, i.e., reversions.

Recently, attention has focused on lentiviral vectors such as those based upon the primate lentiviruses, e.g., human immunodeficiency viruses (HIV) and simian immunodeficiency virus (SIV). Lentiviral vectors are attractive vectors for gene therapy due to their ability to infect and integrate into quiescent cells in addition to dividing cells. Furthermore, lentiviral vectors do not elicit an immune response once integrated. Moreover, by using a pseudotyped vector (i.e., one where an envelope protein from a different species is used), problems encountered with infecting a wide range of cell types can be overcome by selecting a particular envelope protein based upon the cell you want to infect. Moreover, in view of the complex gene splicing patterns seen in a lentiviruses such as HIV, multivalent vectors (i.e., those expressing multiple genes) having a lentiviral core, such as an HIV core, are expected to be more efficient.

Despite the advantages that lentiviral based vectors offer, there is still a significant safety concern with the use of HIV vectors in view of the severity of HIV infection. A prime requirement for successful gene therapy is the sustained expression of the therapeutic gene without any adverse effect on the host. A major concern in using lentiviral-based vectors for gene therapy is the possibility of generating replication-competent lentiviruses during viral production. The major route for generating replication-competent lentiviruses would be through homologous recombination events occurring among the plasmid constructs during transfection.

Much attention has focused on the use of HIV-based vector systems. Because HIV-1 is a known human pathogen, the possibility of generating a replication-competent recombinant virus remains a serious concern.

Accordingly, providing additional attenuated lentiviral vectors that are able to infect a host cell with high efficiency (including resting as well as dividing cells) and have a reduced threat of being a human pathogen would be highly desirable.

### SUMMARY OF THE INVENTION

We have now discovered a SIV-derived vector system wherein multiple lentiviral vectors are used to transfer a heterologous nucleic acid segment to host cells. The SIV-derived vectors are derived from Simian Immunodeficiency Virus (SIV). SIV is not a known human pathogen. The vector system preferably comprises an attenuated SIV virus to create a transfer vector containing the nucleic acid segment of interest, at least one attenuated SIV virus derived packaging vector encoding the proteins and sequences necessary to package the nucleic acid segment into the lentiviral particles, and an env vector encoding an envelope protein from a non-SIV virus, preferably a non-lentivirus. The packaging virus is derived from SIV with at least one of the accessory protein genes (vif, vpr, vpx, and/or nef) deleted. Preferably, the transfer vector and packaging vector are derived from SIVmac. More preferably, SIVmac1A11.

Preferably, the transfer vector contains a 5′ and 3′ lentiviral LTR with the heterologous nucleic acid segment inserted between them, as well as the packaging and leader sequence necessary for encapsidation. Any desired heterologous nucleic acid segment (sometimes referred to as the target molecule) can be inserted into the transfer vector. The nucleic acid segment can be an antisense molecule or more preferably, encodes a protein such as an antibody. The nucleic acid segment can be antisense, a ribozyme, or a gene or gene fragment. Preferably, the heterologous nucleic acid segment is also under the control of a promoter, including an internal, tissue specific, or inducible promoter. It preferably encodes at least a protein. The LTR sequences are preferably based upon SIV. Preferably, the SIV LTR contains a self-inactivating sequence (SIN).

Preferably, the packaging vector is derived from SIVmac1A1 and is deleted for one of the accessory protein genes, vif, vpr, vpx, and nef. More preferably, it is deleted for at least two of the accessory protein genes. Still more preferably, it is deleted for all the accessory genes. More preferably, the SIV packaging vector and the transfer vector do not encode a functional REV/RRE and/or TAT/TAR combination. Preferably, it does not include a functional REV/RRE combination.

Preferably, the env vector contains a nucleic acid sequence encoding an envelope protein operably linked to a promoter is used. This env vector also does not contain a lentiviral packaging sequence.

Preferably these lentiviral vectors contain a selectable marker. These are well known in the art and include genes that change the sensitivity of a cell to a stimulus such as a nutrient, an antibiotic, etc. Genes include those for neo, puro, tk, multiple drug resistance (MDR), etc. Other genes express proteins that can readily be screened for such as green fluorescent protein (GFP), blue fluorescent protein (BFP), luciferase, LacZ, nerve growth factor receptor (NGFR), etc. The nucleic acid segment can be antisense, a ribozyome, or a gene or gene fragment.

In one preferred embodiment, the transfer vector is an SIVmac-derived transfer vector. In a further preferred embodiment, the SIVmac-derived transfer vector does not contain RRE. Instead, a constitutive transport element such as the post-transcriptional control element from spleen necrosis virus LTR is used.

In another preferred embodiment, the vector system comprises two packaging vectors, one of which encodes the gag protein and the other of which encodes at least part of the pol protein.

In another preferred embodiment, the packaging vector has both the rev gene and RRE deleted (i.e., rendered non-functional), and contains the 5′ LTR of spleen necrosis virus. In still another embodiment, the TAT and/or TAR has been deleted (i.e., rendered non-functional).

When an inducible promoter is used with the target molecule, minimal selection pressure is exerted on the transformed cells for those cells where the target molecule is “silenced”. Thus, identification of cells displaying the marker also identifies cells that can express the target molecule. If an inducible promoter is not used, it is preferable to use a “forced-expression” system where the target molecule is linked to the selectable marker by use of an internal ribosome entry site (IRES) [see Marasco et al., PCT/US96/16531].

Preferably, the target molecule is operably linked to an inducible promoter. Such systems allow the careful regulation of gene expression. See Miller, N. and Whelan, J., Human Gene Therapy, 8: 803-815 (1997). Such systems include those using the lac repressor from E. coli as a transcription modulator to regulate transcription from lac operator-bearing mammalian cell promoters and those using the tetracycline repressor (tetR).

Preferably the target molecule contains a tag such as HA so the molecule can be identified later.

Thereafter the lentiviral vectors are used to transduce a host cell. One can rapidly select the transduced cells by screening for the marker. Thereafter, one can take the transduced cells and grow them under the appropriate conditions or insert those cells e.g. spleen cells or germ cells, into a host animal.

### BRIEF DESCRIPTION OF THE DRAWINGS

FIGS. 1A-C show schematics of the elements of an SIVmac-derived lentiviral vector. FIG. 1A depicts SIVpack(−r,−n), a vpr- and nef-defective packaging construct based on SIVmac1A11, which was described previously (White, Renda et al. 1999), and is used as parental construct. This construct contains simian virus-40 early promoter (SV40 E) and poly-adenylation [p(A)] sequences, and a deletion in the SIV packaging element (SIV Ψ). Derivatives of SIVpack(−r,−n) with various combinations of mutations in vif(−v), vpx (−x), vpr (−r), and/or nef(−n) are listed. RRE, Rev-responsive element. FIG. 1B is a schematic of SIVmac1A11-derived transfer vectors. DSIV-1 and DSIV-1ΔRRE contain all cis-acting elements needed for reverse transcription, integration and viral gene expression, including an internal human cytomegalovirus immediate-early promoter (CMV), and differ in the presence of the RRE. DSIV-1 and DSIV-1ΔRRE do not encode any SIV proteins. LTR, long terminal repeat. FIG. 1C depicts construction of packaging constructs for the study of RRE/Rev function. Deletions of RRE and/or rev were introduced as indicated. The spleen necrosis virus (SNV) contains a post-transcriptional control element within the R/U5 region (Butsch, Hull et al. 1999). The entire LTR from SNV was included, which contains viral promoter sequences in the U3 region, thereby replacing the SV40 promoter.

FIG. 2 shows a comparison of viral titers using homologous versus heterologous packaging of RNA. A packaging construct derived from SIVmac1A11. SIVpack(−r,−n) or from HIV-1, pCMVΔR8.2 was co-transfected with a transfer vector and HCMV-VSVG, to produce various lentivirus vectors. DSIV-1 is an SIVmac-derived transfer vector and HIV-GFP is an HIV-1-derived transfer vector (White. Renda et al. 1999). LNCX-GFP is a murine leukemia virus-derived transfer vector (White, Renda et al. 1999), and Ψ(−)env(−)ampho is a packaging plasmid expressing Gag/pol from MLV (Landau and Littman 1992). Indicated plasmids were used to produce vector supernatants, which were concentrated by ultracentrifugation and used for infection of HeLa cells. Infection was measured by GFP fluorescence using flow cytometry. Results are representative of three independent experiments.

FIG. 3 shows the role of RRE/Rev in the Slymac vector system and replacement with the SNV 5′ LTR. The transfer and packaging plasmids used in these experiments were described in FIG. 1. Various combinations of transfer and packaging plasmids with modifications in the RRE or RRE/Rev were co-transfected with HCMV-VSVG to produce vectors as described in FIG. 2. Vectors were used to infect HeLa cells. Resulting titers are plotted and also shown numerically. Results are representative of three independent experiments.

### DETAILED DESCRIPTION OF THE INVENTION

We have now discovered a SIV-derived vector system wherein multiple SIV-derived lentiviral vectors are used to transfer a heterologous nucleic acid segment to host cells. The SIV-derived vectors are derived from Simian Immunodeficiency Virus (SIV). SIV is not a known human pathogen and thus should have additional safety benefits. This system can use any SIV strain including SIVmac, SIVagm, and SIVmnd. Preferably, SIVmac. It is preferred that the virus is an attenuated virus. Many are known and include nef-defective SIV. One can also obtain attenuated strains by passaging the virus and selecting for such strains. For example, SIVmac1A11 is a naturally attenuated lentivirus which is not known to be a human pathogen.

The vector system comprises a SIV-derived transfer vector containing a heterologous nucleic acid sequence (i.e. the nucleic acid segment of interest), a SIV-derived packaging vector encoding the proteins and sequences necessary to package the nucleic acid segment into the lentiviral particles and an env vector encoding an envelope protein from a virus other than SIV. Preferably, the env protein is not from a lentivirus. The transfer vector and the packaging vector is derived from SIV. Preferably, from SIVmac. Further, at least one of the accessory protein genes (vif, vpr, vpx, and/or nef) is deleted.

The heterologous nucleic acid segment, also referred to sometimes as the target molecule, can be any molecule whose expression in a host cell is desired. The target molecules can be proteins such as growth factors, receptors and cytokines, peptides, antibodies, and antisense molecules. Preferably the target molecules are genes encoding proteins. More preferably the proteins are operably linked to an inducible promoter. The vector system can be used to transduce a plurality of cells. Preferably, the vectors contain a marker gene to permit rapid identification and selection of transformed cells.

Numerous modifications can be made to the vectors, which are used to create the particles to further minimize the chance of obtaining wild type revertants. These include deletions of the U3 region of the LTR which includes TAR, tat deletions, RRE deletions, rev deletions and matrix (MA) deletions. The packaging and env vectors contain nucleotides from SIV that package RNA, referred to as the SIV packaging sequence.

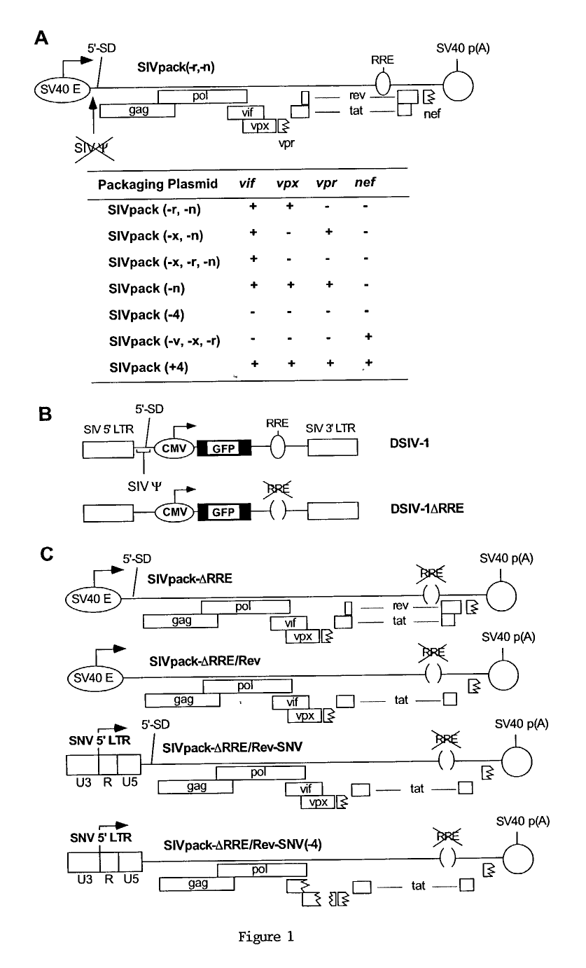

One can use constitutive transport elements (CTE) in place of RRE, to make either the transfer vector or the packaging vector REV independent. For example, one can use the spleen necrosis virus (SNV) LTR, which contains a post-transcriptional control element within the R/U5 region (Butsch et al., 1999). The SNV LTR also contains viral promoter sequences in the U3 region, so if the entire SNV LTR is used, it can replace the SV40 promoter (FIG. 1C). Also, there is less sequence homology. Srinivasakumar, S., et al., J. of Virol., 73:9589-9498 (1999); Srinivasakumar, S. et al. J. of Virol., 71:5841-5848 (1997).

The pol/gag nucleic acid segment(s) on the packaging vectors and the env nucleic acid segment on the env vector will when expressed produce an empty lentiviral particle. By making the above-described modifications such as deleting RRE, the rev coding region, the tat coding region, or the U3 region of the LTR, the possibility of a reversion to a wild type virus is reduced to virtually nil.

The lentiviral virion (particle) is expressed by a vector system encoding the necessary viral proteins to produce a virion (viral particle). Preferably, there is at least one vector containing a nucleic acid sequence encoding the lentiviral pol proteins necessary for reverse transcription and integration, operably linked to a promoter. Preferably, the pol proteins are expressed by multiple vectors. There is also a vector containing a nucleic acid sequence encoding the lentiviral gag proteins necessary for forming a viral capsid operably linked to a promoter. Preferably, this gag nucleic acid sequence is on a separate vector than at least some of the pol nucleic acid sequence, still more preferably it is on a separate vector from all the pol nucleic acid sequences that encode pol proteins.

The preintegration complex of lentiviruses, a family of retroviruses which includes the similar immunodeficiency virus (SIV), human immunodeficiency virus (HIV-1 and HIV-2), have been shown to possess nuclear targeting signals which allow these viruses to infect non-dividing cells including macrophages. The capacity of HIV-1 [P. Lewis et al., EMBO J., 11:3053-3058 (1992); M. Burinsky et al., Proc. Natl. Acad. Sci. USA, 89:6580-6584 (1992)] vectors to stably transduce non-dividing cells has been demonstrated in vitro [J. Reiser et al., Proc. Natl. Acad. Sci. USA, 93:15266-15271 (1996)] and also in vivo [L. Naldini et al., Science, 272:263-267 (1996)]. Thus, these vectors are capable of long-term expression.

A second feature of lentiviral based vectors is the ability to manipulate the target cell range by substituting the envelope glycoprotein with envelope proteins from other viruses which confer an extended host range that can be specifically targeted. For example, robust association between the G protein of vesicular stomatitic virus (VSV)-G protein and the HIV-1 virion core allows virus particles to be concentrated without loss of infectivity and has enabled the production of HIV-1 vector stocks with titers of about 10⁹/ml [J. Reiser et al., Proc. Natl. Acad. Sci. USA, 93:15266-15271 (1996); R. Akkina et al., J. Virol., 70:2581-2585 (1996); J. Yee et al. Proc. Natl. Acad. Sci. USA, 91:9564-9568 (1994)]. Such pseudotyping is also possible with SIV.

The vector(s) forming the particle do not contain a nucleic acid sequence from the SIV genome that will express an envelope protein. Preferably, a separate vector that contains a nucleic acid sequence encoding an envelope protein operably linked to a promoter is used. This env vector also does not contain a lentiviral packaging sequence. In one embodiment the env nucleic acid sequence encodes a lentiviral envelope protein other than an SIV env. Preferably, the envelope protein is not from a lentivirus, but from a different virus. The resultant particle is referred to as a pseudotyped particle. By appropriate selection of envelopes one can infect virtually any cell. For example, one can use an env gene that encodes an envelope protein that targets an endocytic compartment such as that of orthomyxoviruses (influenza virus). VSV-G, alpha viruses (Semliki forest virus, Sindbis virus), arenaviruses (lymphocytic choriomeningitis virus), flaviviruses (tick-borne encephalitis virus, Dengue virus), and rhabdoviruses (vesicular stomatitis virus, rabies virus).

Other envelopes that can preferably be used include those from Moloney Leukemia Virus such as MLV-E, MLV-A and GALV. These latter envelopes are particularly preferred where the host cell is a primary cell. Other envelope proteins can be selected depending upon the desired host cell. For example, targeting specific receptors such as dopamine receptor for brain delivery. Another target can be vascular endothelium. These cells can be targeted using a filovirus envelope. For example, the GP of Ebola, which by post-transcriptional modification become the GP₁ and GP₂ glycoproteins.

A desired family of heterologous nucleic acid segments (sometimes referred to as the target molecules) can be inserted into the empty SIV-derived particles by use of a transfer vector containing a nucleic acid segment of interest and a SIV packaging sequence necessary to package SIV RNA into the lentiviral particles. Preferably, the transfer vector contains a 5′ and 3′ lentiviral LTR with the desired nucleic acid segment inserted between them. The nucleic acid segment can be an antisense molecule or more preferably, encodes a protein. The packaging vector preferably contains a selectable marker. These are well known in the art and include genes that change the sensitivity of a cell to a stimulus such as a nutrient, an antibiotic, etc. Genes include those for neo, puro, tk, multiple drug resistance (MDR), etc. Other genes express proteins that can readily be screened for such as green fluorescent protein (GFP), blue fluorescent protein (BFP), luciferase, LacZ, nerve growth factor receptor (NGFR), etc.

As used herein, the introduction of DNA into a host cell is referred to as transduction, sometimes also known as transfection or infection.

One can set up systems to screen cells automatically for the marker. In this way one can rapidly select transduced cells from non-transduced cells. For example, the resultant particles can be contacted with about one million cells. Even at transduction rates of 10-15% one will obtain 100-150.000 cells. An automatic sorter that screens and selects cells displaying the marker, e.g. GFP, can be used in the present method.

When an inducible promoter is used with the target molecule, minimal selection pressure is exerted on the transformed cells for those cells where the target molecule is “silenced”. Thus, identification of cells displaying the marker also identifies cells that can express the target molecule. If an inducible promoter is not used, it is preferable to use a “forced-expression” system where the target molecule is linked to the selectable marker by use of an internal ribosome entry site (IRES) (see Marasco et al., PCT/US96/16531). In this manner, virtually all cells selected on the basis of the marker also contain and can express the target molecule.

IRES sequences are known in the art and include those from encephalomyocarditis virus (EMCV) [Ghattas, I. R. et al., Mol. Cell. Biol., 11:5848-5849 (1991)]; BiP protein [Macejak and Sarnow, Nature, 353:91 (1991)]; the Antennapedia gene of Drosophila (exons d and e) [Oh et al., Genes & Development, 6:1643-1653 (1992)]; those in polio virus [Pelletier and Sonenberg, Nature, 334:320-325 (1988); see also Mountford and Smith, TIG, 11: 179-184 (1985)].

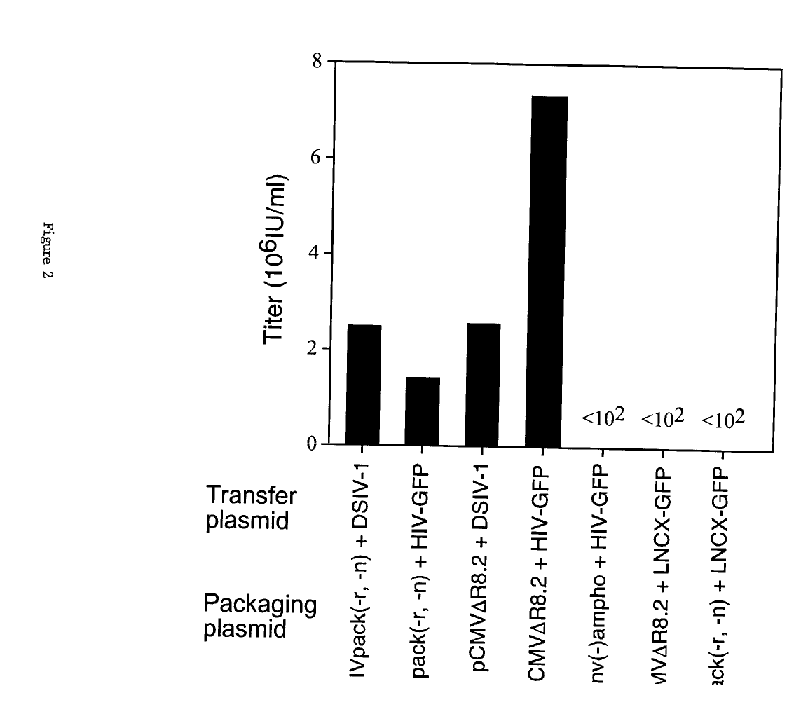

Preferably, the target molecule is operably linked to an inducible promoter. Such systems allow the careful regulation of gene expression. See Miller, N. and Whelan, J., Human Gene Therapy, 8:803-815 (1997). Such systems include those using the lac repressor from E. coli as a transcription modulator to regulate transcription from lac operator-bearing mammalian cell promoters [Brown, M. et al., Cell, 49:603-612 (1987)], and those using the tetracycline repressor (tetR) [Gossen, M., and Bujard H., Proc. Natl. Acad. Sci. USA 89:5-547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using estradiol, RU486, diphenol murislerone or rapamycin [see Miller and Whelan, supra at FIG. 2]. Inducible systems are available from Invitrogen. Clontech and Ariad. Systems using a repressor with the operon are preferred. For example, the lac repressor from Escherichia coli can function as a transcriptional modulator to regulate transcription from lac operator-bearing mammalian cell promoters [M. Brown et al. Cell, 49:603-612 (1987)]. M. Gossen et al. [Proc. Natl. Acad. Sci. USA, 89:5547-5551 (1992)] combined the tetracycline repressor (tetR) with the transcription activator (VP16) to create a tetR-mammalian cell transcriptional activator fusion protein, tTa (tetR-VP16), with the tetO-bearing minimal promoter derived from the human cytomegalovirus (hCMV) major immediate-early promoter to create a tetR-tet operator system to control gene expression in mammalian cells. The tetracycline repressor (tetR) alone, rather than the tetR-mammalian cell transcription factor fusion derivatives can function as potent trans-modulator to regulate gene expression in mammalian cells when the tetracycline operator is properly positioned downstream of the TATA element of a promoter such as the CMVIE promoter [Human Gene Therapy, 11:577-585 (2000)]. One particular advantage of this tetracycline inducible switch is that it does not require the use of a tetracycline repressor-mammalian cell transactivator or repressor fusion protein, which in some instances can be toxic to cells [M. Gossen et al., Proc. Natl. Acad. Sci. USA 89:5547-5551 (1992); P. Shocken et al., Proc. Natl. Acad. Sci. USA 92:6522-6526 (1995)], to achieve its regulatable effects.

The effectiveness of some inducible promoters increases over time. In such cases one can enhance the effectiveness of such systems by inserting multiple repressors in tandem, e.g. TetR linked to a TetR by an IRES. Alternatively, one can wait at least 3 days before screening for the desired function.

The lentiviral virion (particle) is expressed by at least one vector containing a nucleic acid sequence encoding the lentiviral pol and gag proteins necessary for viral protein expression operably linked to a promoter. Preferably, multiple vectors are used. Preferably, the pol sequences encoding pol proteins are on more than one vector. There is also a vector having nucleic acid sequence encoding the lentiviral gag proteins necessary for reverse transcription and integration operably linked to a promoter. Preferably, this gag nucleic acid sequence is on a separate vector than the pol nucleic acid sequence. The use of separate vectors for the various “genes” further reduces the chance of a reversion to wild-type.

In one embodiment, the lentiviral vector is modified so that the gag sequence does not express a functional MA, protein, i.e. it is MA⁻. This can be accomplished by inactivating the “gene” encoding the MA by additions, substitutions or deletions of the MA coding region. Since the MA is part of the gag gene and as expressed, is processed from the precursor protein, when referring to a MA gene (or coding region), we are only referring to that portion of the entire gag gene that encodes the MA subunit. Preferably, the inactivation is accomplished by deletion.

The MA has a myristylation anchor and that myristylation anchor (sequence) is required. Preferably, the myristylation sequence is a heterologous (i.e., non-lentiviral) sequence. Src, MARCKS (myristolylated alanine-rich C kinase substrate), ARF (ADP-ribosylation factor), recovering and related EF-hand calcium-binding proteins (visinin neurocalcium and others), and non-lentiviral gag proteins (e.g., Moloney murine leukemia virus, Mason-Pfizer monkey virus).

In another embodiment the SIV derived vector is another form of self-inactivating (SIN) vector as a result of a deletion in the 3′ long terminal repeat region (LTR). Preferably, the vector contains a deletion within the viral promoter. The LTR of lentiviruses such as the SIV LTR contains a viral promoter. Although this promoter is relatively inefficient, when transactivated by e.g. tat, the promoter is efficient. The SIV LTR promoter is preferably deleted.

Preferably, the vector contains a deletion within the viral promoter. The viral promoter is in the U3 region of the 3′ LTR. The further 5′ you go the more dramatic the “SIN” effect is. After reverse transcription, the deletion is transferred to the 5′ LTR, yielding a vector/provirus that is incapable of synthesizing vector transcripts from the 5′ LTR in the next round of replication. Thus, the vector of the present invention contains no mechanism by which the virus can replicate as it cannot express the viral proteins.

In another embodiment the vector is a tat deleted vector. This can be accomplished by inactivating at least the first exon of tat by known techniques such as deleting it. Alternatively, one can extend the U3 LTR deletion into the R region to remove the TAR element. The tat deleted vectors result in high titer of virus.

In a preferred embodiment, the packaging and transfer vectors have multiple modifications as compared to a wildtype virus. For example, with SIV being nef-, vpx-, vif- and vpr-.

In a more preferred embodiment, the env vector contains a gene encoding an envelope protein from influenza virus or VSV, more preferably VSV-G.

While env glycoproteins are dispensable for particle production per se, their incorporation is required for the formation of infectious virions.

The vector system can be used to package a wide range of desired nucleotide segments, preferably a RNA segment, into an empty lentiviral particle because of the relatively large genomes of lentiviruses. In addition, the use of promoters and enhancers can also significantly add to the length of an insert.

The packaging vector(s) is prepared so that none of the nucleotide segments used will contain a functional packaging site containing sequence. (This sequence is referred to as the packaging sequence.) Preferably, these vectors also do not have lentiviral LTRs such as the SIV LTR. The env, gag and pol genes are operably linked to a heterologous promoter. A preferred promoter is the SV40 early promoter (SV40E) (see FIGS. 1A and C). Preferably, the packaging vector(s) also contains a polyadenylation sequence, such as the polyadenylation sequence from SV40 (see FIGS. 1A, C).

The packaging sequence can be excluded from the vector(s) by any of a variety of techniques well known to the person of ordinary skill in the art. For example, one can simply delete the entire sequence. Alternatively, one can delete a sufficient portion of a sequence to render it incapable of packaging. An alternative strategy is to insert nucleotides into such a site to render it non-functional. Most preferably, one will delete the site entirely to prevent homologous recombination.

Accordingly, packaging lentiviral vectors can express the desired viral proteins, but because the packaging site has been removed, and the lentiviral LTRs are not operational their mRNA will not be effectively packaged into the lentiviral particles, and the recombinant virus will not be able to replicate and infect other cells.

The packaging vectors can also contain sequences encoding desired lentiviral regulatory proteins such as Tat, Rev, etc. However, in a number of embodiments it is preferable not to contain such regulatory genes. If RRE sequences are included in the gene, the inclusion of sequence encoding rev is necessary, unless the virus is expressed in the cytoplasm. These regulatory sequences can be on a separate packaging vector (e.g., gag vector, pol vector, gag-pol vector, or env, vector), or on their own lentiviral vector.

A desired heterologous nucleic acid segment can be encapsulated within the empty lentiviral particle by using a transfer vector containing a nucleic acid segment of interest and a SIV packaging sequence necessary to package SIV RNA into the SIV particles at the time the SIV transfer vectors are used. Preferably, the transfer vector contains a 5′ and 3′ lentiviral LTR with the desired heterologous nucleic acid segment inserted between them. Preferably, the heterologous nucleic acid segment encodes a protein.

Accordingly, as used herein, the transfer vector refers to the SIV-derived vector that contains the heterologous gene to be transferred under the control of a promoter (e.g., internal, tissue specific, or inducible) flanked by SIV LTRs, and the SIV packaging and leader sequence necessary for encapsidation (i.e., packaging). The packaging vector encodes the proteins and enzymes required for encapsidation. See FIG. 1.

The SIV-derived transfer vector may also contain an origin of DNA replication (ori) which is recognized by the viral replication proteins such as the simian virus 40 large T antigen, present in the virus-producer cells, such as 203-T or COS-7 cells. This transfer vector permits packaging of desired nucleotide inserts in the pseudotyped particles. This transfer vector is used to package any group of desired heterologous nucleic acid sequence, preferably a RNA sequence, into the particle. Preferably, the transfer vector contains (a) a promoter sequence operably linked to at least one heterologous nucleic acid sequence and (b) at least one sequence sufficient to permit transcription and processing of mRNA, the translation of which results in an expressed protein. Preferably, the processing sequence is a polyadenylation sequence. Preferably the promoter is part of an inducible system. Still more preferably, this transfer vector contains an intervening sequence following the promoter sequence. Preferably the sequences containing the promoter, target molecule, and optionally a repressor sequence also contains a tag such as HA to permit ready identification of the target molecule. This grouping of elements is sometimes also referred to as the cassette. For example, the heterologous sequence can encode any desired protein, preferably a therapeutic protein or an antibody. It can also encode antisense DNA, RNA or a desired immunogen, such as an antigenic protein. It can encode specific peptide sequence that will generate an immunogenic reaction. Such a peptide sequence is typically at least about 6 amino acids in length.

The heterologous nucleotide sequence can encode a wide variety of proteins such as a therapeutic protein, i.e., one that compensates for an inherited or acquired deficiency. Examples of therapeutic proteins include neurotransmitter biosynthetic enzymes, e.g., tyrosine hydroxylase for the treatment of Parkinson's disease; neurotrophic factors including neutrophins, e.g. nerve growth factor for the treatment of Alzheimer's disease, one can also use nerve growth factor receptor and the trk receptor; hypoxanthine-guanine porphoribosyl transferase (HGPRT) for the treatment of Lesch Nyhan disease; β-hexosaminadase α chain for the treatment of Tay Sachs disease; insulin for the treatment of diabetes. Receptors can also be prepared, e.g. the nerve growth factor receptor, the trk receptor, etc. Because the insert can be large, it is possible to encode a series of different proteins. For example, one can encode a series of proteins that form a receptor-ligand complex.

Other proteins include, for example, signal transduction enzymes, e.g. protein kinase c: transcription factors, e.g., c-fos, NF-PB; oncogenes, e.g. erbB, erbB-2/neu, ras; neurotransmitter receptors, e.g., glutamate receptor, dopamine receptor, etc.

The heterologous nucleotide sequence can also encode antisense molecules (DNA or RNA). These molecules can be used to regulate gene expression associated with a particular disease. The antisense molecules are obtained from a nucleotide sequence by reversing the orientation of the coding region with regard to the promoter. Thus, the antisense RNA is complementary to the corresponding mRNA. For review of antisense science see Green, et al., Ann. Rev. Biochem. 55: 569-597 (1986), which is herein incorporated by reference. Another class of molecule includes ribozymes. Ribozymes and antisense molecules that engage in, as well as those that do not show transplicing can be used.

The heterologous nucleotide sequence is preferably operably linked to a promoter sequence capable of directing transcription of the sequence in a desired target cell. Lentiviruses such as the primate lentiviruses contain the Tat regulatory protein. This protein will transactivate a protein operably linked to a TAR element. The TAR element is present in the 5′ LTR of the primate lentivirus. Thus, the expression of heterologous protein can be enhanced by transactivation. The LTR also contains a promoter. However, that promoter in the absence of transactivation is relatively ineffective. Thus, the use of other promoters and enhancers is typically preferred. The promoter can be a promoter such as the SV40, CMV, HSV-1 IE, IE 4/5 or RSV (Rous sarcoma virus) promoters. The CMV promoter is a preferred promoter. Others include Srα-promoter (a very strong hybrid promoter composed of the SV40 early promoter fused to the R/U5 sequences from the HTLV-I LTR), tetracycline-regulatable promoters, tissue-specific promoters (e.g., alpha-fetoprotein promoter: and rhodopsin promoter for photoreceptor-targeted expression). Other promoters capable of directing transcription of the heterologous sequence in a specific target cell can also be used to more specifically direct expression of the heterologus gene to a desired target (host) cell. Indeed, one can link the inducible promoter construct with a tissue specific promoter. For example, if the target cell is a neuronal cell, a promoter such as the neuron specific enolase promoter [Forss-Petter, et al., J. Neurosci. Res. 16: 141-56 (1986)] can be used. The rat tyrosine hydroxylase (TH) promoter can support cell type specific expression in the midbrain [S. Song et al., J. Neurochem. 68: 1792-803 (1997)].

In order to minimize the possibility of a recombination event between the transfer vector that transfers the desired heterologus gene(s) and the packaging vector, generating a wild type lentivirus, it is desirable that the transfer vector has a minimal degree of homology with the nucleotide segments encoding the packaging vector. Preferably, one would use different promoters in these different vectors. These goals can be accomplished by a variety of means known in the art based upon the present disclosure. For example, in order to minimize any chance of recombination, it is preferable to use multiple vectors. Additionally, it is preferable to reduce the chance of homologous recombination by minimizing sequence overlap. For example, one can delete unnecessary lentiviral sequences. Alternatively or additionally, one can use known techniques to change the nucleotide sequence of the vectors. One method of accomplishing this is referred to as nucleotide, e.g., DNA, shuffling. One changes nucleotides in codons, e.g., the third base of each codon within the lentiviral constructs of one vector. Thus, the same coding sequence in a second vector now differs and will not be subject to homologous recombination. Changes in the codons of the various vectors can be made to optimize nucleotide differences.

Alternatively or in combination with the above approach of reducing homology, one can alter the sequence of a gene from the lentivirus segment so that it does not encode a functional protein. As used herein “functional” means a protein having wild-type activity.

Depending upon the particular purpose for the particles one can use known techniques to alter the lentivirus segment to inactivate genes that encode proteins present in the particle which cause certain effects. For example, inactivating those proteins that enhance replication, e.g., rev and/or tat. Nef also affects the virus. It has been reported that nef appears to be required for efficient replication in vivo.

Cells can be transfected by the vectors to prepare the viral particle. One can prepare the vectors in vitro, one would then harvest the particles, purify them and inject them by means well known in the art. More preferably one would purify the particles, and then use those to infect the desired cells.

One can create producer cell lines expressing virions and transform such cells with the packaging vector. The producer cell lines or any cell can be transformed by standard techniques. One preferred method is to use an inactivated adenovirus vector linked to the packaging vector by a condensing polycation such as polylysine or polyethylanimine (PEI) [see Baker, A. et al., Nucleic Acids Res., 25(10):1950-1956 (1997); Baker, A. et al., Gene Ther., 4(8):773-782 (1997); Scaria, A. et al., Gene Ther., 2:295-298 (1995)]. The use of PEI as a condensing polycation is preferred.

The vectors express proteins and mRNA which assemble into particles and hence can be used to express large amounts of viral particles. This requires transfecting a cell with the particle vector system described herein, the packaging vector, and culturing the cell line under conditions and time sufficient to express the viral proteins, which then form the particles. Thereafter, the particles can be purified by known techniques with care taken to insure that the structure of the particle is not destroyed. The particles can be used in a variety of areas. For example, they can be used to generate a desired immune reaction, to transform a cell with a heterologous nucleic acid sequence and/or to deliver a nucleic acid sequence to a desired host cell.

One can prepare transient or stable cell lines that express the lentiviral particles by standard techniques based upon the present teaching.

Thereafter, if stable cell lines are desired, one can screen for those cells that have been stably transfected by standard technique.

Such stable producer cell lines are a preferred source for obtaining packaged particles.

The particles of the present invention can be used to deliver heterologous DNA to a target cell. The target cell may be in vivo, in vitro or ex vivo. The target cell can be a dividing or preferably a quiescent cell. Quiescent cells include nonmitotic or postmitotic cells. The preferred nonmitotic cell is a macrophage. The target cells also include cells of the nervous system, e.g., neural or neuronal cells. Preferred quiescent or slowly dividing target cells include glia cells, myocytes, hepatocytes, pneumocytes, retinal cells, and hematopoietic stem cells. Pancreatic islet cell are also a preferred target.

In the present method the use of in vitro cells in presently preferred. However, there are instances where in vivo or ex vivo administration is desirable.

Introduction of the viral particle carrying the heterologous gene to be delivered to a target cell may be effected by any method known to those of skill in the art. For example, with in vivo administration, the following techniques are preferred. Catheters, injection, scarification, etc. For example, stereotaxic injection can be used to direct the viral particles to a desired location in the brain. Stereotaxic surgery is performed using standard neurosurgical procedures [Pellegrino and Clapp, Physiol. Behav. 7: 863-8 (1971)]. Additionally, the particles can be delivered by intracerebroventricular (“icv”) infusion using a minipump infusion system, such as a SynchroMed Infusion System. A recent method based on bulk flow, termed convection, has also proven effective at delivering large molecules to extended areas of the brain and may be useful in delivering the viral particle to the target cell [R. Bobo et al., Proc. Natl. Acad. Sci. USA 91: 2076-80 (1994); P. Morrison et al., Am. J. Physiol. 266: R292-305 (1994)]. Other methods can be used including catheters, intravenous, parenteral, intraperitoneal and subcutaneous injection, oral or other known routes of administration.

In some instances one would use these vectors to transform host cells in vivo to look for the effects of specific genes in a living system. One would inject a sufficient amount of the separate vectors or preferably the packaged viral particles to obtain a serum concentration in the tissue containing the target cell of the therapeutic protein ranging between about 1 pg/ml to 20 μg/ml. For example, by expressing a specific protein or, alternatively stopping the function of a protein such as by expressing an antibody to a specific sequence intracellularly. More preferably between about 0.1 μg/ml to 10 μg/ml. Still more preferably, between about 0.5 μg/ml to 10 μg/ml.

For example, solid dose forms that can be used for oral administration include capsules, tablets, pills, powders and granules. In such solid dose forms, the active ingredient, i.e., empty virus particle, is mixed with at least one inert carrier such as sucrose, lactose or starch. Such dose forms can also comprise additional substances other than inert diluents, e.g., lubricating agents, such as magnesium stearate. Furthermore, the dose forms in the case of capsules, tablets and pills may also comprise buffering agents. The tablets, capsules and pills can also contain time-release coatings to release the particles over a predetermined time period.

For parenteral administration, one typically includes sterile aqueous or nonaqueous solutions, suspensions or emulsions in association with a pharmaceutically acceptable parenteral vehicle. Examples of non-aqueous solvents or vehicles are propylene glycol, polyethylene glycol, vegetable oils such as olive oil and corn oil, gelatin and injectable organic esters, such as ethyl oleate. These dose forms may also contain adjuvants such as preserving, wetting, emulsifying and dispersing agents. They may be sterilized by, for example, filtration through a bacterial-retaining filter, by incorporating sterilizing agents into the composition, by irradiating the compositions, etc, so long as care is taken not to inactivate the virus particle. They can also be manufactured in a medium of sterile water or some other sterile injectable medium before use. Further examples of these vehicles include saline, Ringer's solution, dextrose solution and 5% human serum albumin. Liposomes may also be used as carriers. Additives, such as substances that enhance isotonicity and chemical stability, e.g., buffers and preservatives, may also be used.

The preferred range of active ingredient in such vehicles is in concentrations of about 1 mg/ml to about 10 mg/ml. More preferably, about 3 mg/ml to about 10 mg/ml.

### EXAMPLES

The construction of a minimal SIV packaging plasmid is described, wherein accessory genes have been deleted. Our results indicate that deletion of the accessory genes had minimal or no effect on viral titers or on the ability to transduce dividing and aphidicolin-arrested cells. We constructed an SIVmac-based transfer vector using the SIVmac1A11 strain. SIVmac1A11 is a non-virulent molecular clone which causes self-limiting, non-pathogenic infection in rhesus macaques (Marthas, Sutjipto et al. 1990). These vectors can transduce dividing and non-dividing cells with similar abilities. The reciprocal packaging of an SIVmac-based transfer vector with an HIV-1-based packaging construct was also examined.

Finally, we explored the requirement for the RRE/Rev system in both the packaging and the transfer plasmids, and tested the possibility of its replacement using the spleen necrosis virus (SNV) 5′ LTR.

Materials and Methods

Cell Lines

The human embryonic 293T cells and human T-cells, CEM, were propagated in Iscove's modified Dulbecco's medium (IMDM; BioWhittaker. Walkersville, Md.) plus 10% fetal calf serum (FCS).

Plasmid Construction

SIVmac1A11 proviral sequences were obtained from the plasmid pSVT3/1A11 (Marthas, Sutjipto et al. 1990). The full sequence of SIVmac1A11 is available through GenBank (Accession number M76764). Construction of the vectors, SIVpack(−r,−n), originally named SIVpack, and HIV-GFP was described previously (White, Renda et al. 1999).

A 110-base-pair fragment of vpx, containing codons glu58 to arg109 was deleted by PCR mutagenesis from SIVpack(−r, −n) to produce SIVpack(−x, −r, −n). Full-length vpr was restored into SIVpack(−x, −r, −n) by cloning an Nco I to Sph I fragment of SIVmac239 (Regier and Desrosiers 1990) to generate SIVpack(−x, −n). The same Nco I-to-Sph I fragment containing vpr was cloned into SIVpack(−r, −n) to give rise to SIVpack(−n). SIVpack (−x, −r, −n) was digested with BstB I and Sac 1, treated with Mung bean nuclease, and religated, and this resulted in truncation of vifat codon arg139, to give rise to SIVpack(−4). Nef was restored in SIVpack(−n) by subcloning a restriction fragment comprised between Sal I and PflM I from pSVT3/1A11 (Marthas, Sutjipto et al. 1990), between SalI and BsaBI. This resulted in restoration of full-length, wild-type nef gene from SIVmac1A11 (Unger, Marthas et al. 1992) to produce SIVpack (+4). Similarly, full-length nef contained between the Sal I and PflM I (treated with Mung bean nuclease for blunting) sites of pSVT3/1A11 was inserted between the Sal I and BsaB I sites in SIVpack(−4), to produce SIVpack(−v, −x, −r).

For construction of the SIV-based transfer vectors, pSVT3/1A11ΔSpe was used as the parental construct. pSVT3/1A11ΔspeI was constructed by digesting pSVT3/1A11, with Spe I and religating. This resulted in deletion of the SIVmac1A11 sequences comprised between nucleotides 1775 and 7040 (Genbank accession M76764). A linker composed of the oligonucleotides 5′-CGATGCGGCCGCAAAAGGAAAAA-3′ and 5′-CTAGTTTTCCTTTTGCGGCCGCAT-3′ was then cloned between the Cla I and Spe I sites of pSVT3/1A11ΔSpe, to generate the construct pSVT4-LNK#4. A fragment containing the CMV promoter and eGFP was subcloned from pEGFP-N1 (Clontech. Palo Alto, Calif.) using Not I and Spe I, into pSVT4-LNK#4, which was digested with Not I and Afl III. In this cloning, the Spe I and Afl III ends were treated with the Klenow fragment of DNA polymerase I. The SV40 promoter was then inactivated by deleting a fragment comprised between Drd I and Sph I, treating with Mung bean nuclease, and religating the vector. The resulting plasmid was DSIV-1. DSIV-1ΔRRE was constructed replacing a fragment comprised between Not I and Sal I (which contained the RRE) in DSIV-1 with a linker from the following oligonucleotides: 5′-TCGACGTCGGCCATAGGC-3′ and 5′-GGCCGCCTATGGCCGACG-3′.

For construction of SIVpack-ΔRRE-SNV, the SV40 promoter was eliminated by deleting a fragment comprised between Drd I and Sph I, treating with Mung bean nuclease, and religating the vector, to produce the intermediate clone, SIVpack-ΔSV40pro. The SNV 5′-LTR fragment between Nar I and Hind III was subcloned from pKB402 (Butsch, Hull et al. 1999), into SIVpack-ΔSV40pro using Nar I and Hind III, to produce SIVpack+SNV-LTR. SIVpack+SNV-LTR was digested with Nhe I and Cla 1, treated with Klenow to fill the ends, and religated, to produce SIVpack-ΔRRE-SNV. The second coding exon of rev was then deleted by digestion of SIVpack-ΔRRE-SNV with Nhe I and Mun I, Klenow fill-in, and relegation, to yield SIVpack-ΔRRE/Rev-SNV.

Deletion of Rev and RRE from the parent packaging plasmid, SIVpack (−r, −n) was accomplished by digestion with Nhe I and Cla I, treatment with Klenow to fill the ends, and relegation, to produce SIVpack-ΔRRE. The second coding exon of rev was then deleted by digestion of SIVpack-ΔRRE with Nhe I and Sal I, Klenow fill-in, and religation, to yield SIVpack-ΔRRE/Rev.

Deletion of the accessory genes in SIVpack-ΔRRE/Rev-SNV was performed by removing the fragment between BstBI and SacI, treating it with Mung bean nuclease, and religating the vector, to generate SIVpack-ΔRRE/Rev-SNV(−4).

Viral Vector Production and Titration

Lentiviral vectors were produced by transient transfection of 293-T cells SIVpack variants or pCMVΔR8.2 and HIV-GFP or DSIV-1 were co-transfected with HCMV-VSVG using the calcium phosphate co-precipitation method. Virus was collected at 48, 72 and 96 hours post-transfection. The harvested supernatant (35 ml) was pre-cleared by low-speed centrifugation at 2.000 rpm, and pelleted by ultra-centrifugation at 25,000 rpm in a Discovery 100S centrifuge with a Surespin 630 rotor (Kendro, Newton, Conn.). Virus pellets were resuspended in 0.5 ml of fresh medium, and frozen at −80 C. Ultracentrifugation resulted in a 70-fold concentration factor, and there was no apparent loss in total vector infectivity. Vector titers were measured by infection (see below), followed by flow cytometric analysis of cells positive for the reporter molecule, GFP. Vector titers were calculated as follows:

Titer=[F×C₀/V]×D

F=frequency of GFP (+) cells by flow cytometry; C₀=total number of target cells at the time of infection: V=volume of inoculum; D=virus dilution factor. Virus dilution factor used for titrations was always D=10. Total number of target cells at the time of infection was 10⁶. Vector titers prior to concentration were between 10⁴ to 10⁵ IU/ml.

All of the vectors described in this work were tested for the emergence of replication-competent lentivirus (RCL) at least once, using a previously described method (White, Renda et al. 1999). We used CEMX174 cells (Stefano, Collman et al. 1993) as indicator cells because they are susceptible to infection by a broad range of SIVmac and HIV-1 strains. 293-T cells that had been transfected with lentiviral vectors, or supernatants thereof were co-cultured with CEMX174 cells (first vector passage) for 48 hours. Supernatants from vector-infected CEMX174 cells were mixed with fresh CEMX174 cells (second vector passage) and then cultured for 14 days. The presence of RCL was evaluated by GFP fluorescence and SIV p27 capture ELISA at day 14 after exposure to second vector passage. RCL was never detected by ELISA or GFP expression. In addition, supernatants from indicator CEMX174 cells at the were used to infect MAGI cells (Kimpton and Emerman 1992) for detection of any potential recombinant viruses which may retain expression of tat. No blue foci could be identified in these cells at 3 days post-exposure. Visual examination of second-passage CEMX174 cells and MAGI cells failed to reveal any cytopathic effects which might have been expected from the presence of replication-competent viruses.

Infections of Dividing and Growth-Arrested CEM Cells

Exponentially-growing CEM cells were treated with 6 μg of aphidicolin (Sigma Chemical Co., St. Louis, Mo.) in DMSO, 24 hours prior to infection, to arrest their growth. Aphidicolin was also maintained in the medium for the first 24 hours post-infection. Infections were performed as follows. Vectors were thawed at 37 C. and 250 μl of vector, 1,250 μl of medium, 15 μg of polybrene (Sigma Chemical Co., St. Louis, Mo.) were mixed with 106 dividing or growth arrested CEM cells. The infection mixture was placed on a rocking platform at 37 C for 2 hours, after which the cells were washed twice with normal medium and cultured until the time of analysis (48 or 72 hours) by flow cytometry.

Flow Cytometry

Cells were harvested and analyzed by direct immunofluorescence for GFP expression. Cells were detached with 2 mM EDTA, washed in phosphate-buffered saline (PBS), fixed with 0.2% paraformaldehyde in PBS for 1 hour. Flow cytometric analysis was performed in an Epics Elite ESP (Coulter Corp., Hialeah, Fla.). Gates for detection of GFP were established using mock-infected cells as background. Because electronic settings varied from experiment to experiment, gates were defined such that percentage of false positive events was not higher than 0.3% in the mock-infected population.

Deletion of Accessory Genes in an SIVmac-Derived Packaging Plasmid

The packaging construct, named SIVpack, was derived from SIVmac1A11. The transfer vector was derived from SIV-1, and the env vector utilized VSV-G. The pathogenesis by lentiviruses is largely attributed to the expression of various open reading frames termed “accessory” genes (Frankel and Young 1998). The effect of removal of accessory genes from this packaging vector on viral titer and on its ability to transduce non-dividing cells was also examined. Various groups (Zufferey, Nagy et al. 1997: Kim, Mitrophanous et al. 1998: Mochizuki, Schwartz et al. 1998; Uchida. Sutton et al. 1998) have shown that HIV-1 derived retroviral particles deleted in all accessory genes (vif, vpu, vpr, nef) can be produced in high titers and preserve the ability to infect non-dividing cells.

The parental construct, SIVpack (White, Renda et al. 1999) is referred to herein as SIVpack(−r, −n), because it contains truncated versions of the vpr (“r”) and nef (“n”) genes. Packaging constructs with various genotypes in the accessory genes were created (FIG. 1A). Such constructs included SIVpack(−4) and SIVpack(+4), in which all accessory genes were excluded or included, respectively. These mutant packaging plasmids were then used in co-transfection with an HIV-1-derived transfer vector, HIV-GFP (White, Renda et al. 1999), and VSV-G, to produce RNA pseudotype lentivirus vectors. The resulting vector supernatants were concentrated by ultracentrifugation and titrated on dividing and aphidicolin-arrested CEM cells. GFP-positive cells were scored by FACS analysis 2 days after transduction. All vector titers presented in this study correspond to concentrated preparations. No significant effect on viral titers or their ability to infect dividing as well as aphidicolin arrested cells was seen (Table 1). In particular, the titers of SIVpack(−4) and SIVpack(+4) was not significantly different (1.51×10⁶ and 1.26×10⁶ IU/ml, respectively).

An SIV-derived transfer vector was constructed. One potential use of SIV transfer vectors, when used in conjunction with SIV packaging vectors, would be the possibility of including a reagent (such as a ribozyme, or an anti-sense gene) directed against HIV-1 sequences. Because of the sequence divergence between HIV-1 and SIVmac, the inhibitory reagent should be effective against HIV-1 but not against the vector itself.

An SIVmac1A11-based transfer vector, DSIV-1, was constructed (FIG. 1, panel B), containing an internal CMV promoter driving GFP expression. DHIV-1 was packaged with various SIVpack mutants and pseudotyped with VSV-G, and titrated on dividing and non-dividing CEM cells (Table 1). The titers obtained with DHIV-1 were similar to those obtained with HIV-GFP (Table 1). This was surprising because we expected that the encapsidation of an SIVmac-derived transfer vector by an SIVmac packaging plasmid would be more efficient than that of HIV-GFP. This may change when used in vivo. The titers of the vectors containing DSIV-1 were also unaffected by the removal of any or all of the SIVmac accessory genes from the packaging construct (Table 1).

Reciprocal Packaging of HIV-1 and SIVmac Vectors

Previous work by Rizvi et al. has shown that SIV-RNA can be efficiently packaged into HIV particles (Rizvi and Panganiban 1992: Rizvi and Panganiban 1993). HIV-1 was also shown to package other non-homologous RNAs such as those from HIV-2 (Arya and Gallo 1996; Poeschla, Corbeau et al. 1996; Kaye and Lever 1998) and SIV (Rizvi and Panganiban 1993). Different combinations of SIVmac and HIV-1-derived elements were used to generate lentivirus vectors. As an HIV-1 packaging plasmid we used pCMVΔR8.2 (Naldini, Blomer et al. 1996). As shown in FIG. 2, efficient reciprocal packaging of HIV-1 and SIVmac1A11 based constructs was possible. The titers produced by the packaging construct, SIV-pack(−r, −n) when using DSIV-1 (homologous packaging) and HIV-GFP were 2.50×10⁶ IU/ml, and 1.41×10⁶ IU/ml, respectively. Vectors packaged with pCMVΔR8.2 produced titers of 7.32×10⁶ IU/ml with HIV-GFP (homologous packaging) and 2.56×10⁶ IU/ml with DSIV-1. Thus, although subtle differences may be noted between homologous and heterologous packaging, these differences do not dramatically affect vector titers or the ability to infect non-dividing cells.

Heterologous packaging of an HIV-1-derived genome by SIVmac proteins, and the converse, are possible due to the relative phylogenetic proximity of SIVmac and HIV-1. A packaging plasmid from a more distantly related virus, such as the murine leukemia virus (MLV), would not be expected to efficiently encapsidate an HIV-1-derived genome. Co-transfection of HIV-GFP and a MLV packaging construct, Ψ(−)env(−)ampho (Landau and Littman 1992), produced no detectable transduction, as judged by GFP expression (<100 IU/ml). Conversely, a murine transfer vector, LNCX-GFP, was also unable to produce detectable transduction when co-transfected with either SIVpack(−r, −n) or pCMVΔR8.2. Thus, encapsidation of a heterologous transfer vector by lentivirus-derived viral particles is specific because it occurs when the transfer and packaging vectors are from closely related viruses but not distantly related ones.

Role of Rev and the RRE in Vector Production

All of the above SIVpack-derived packaging constructs encode both rev and the rev-responsive element (RRE: FIG. 1 panel A). In addition, the DSIV-1 transfer plasmid also contains the RRE (FIG. 1 panel B).

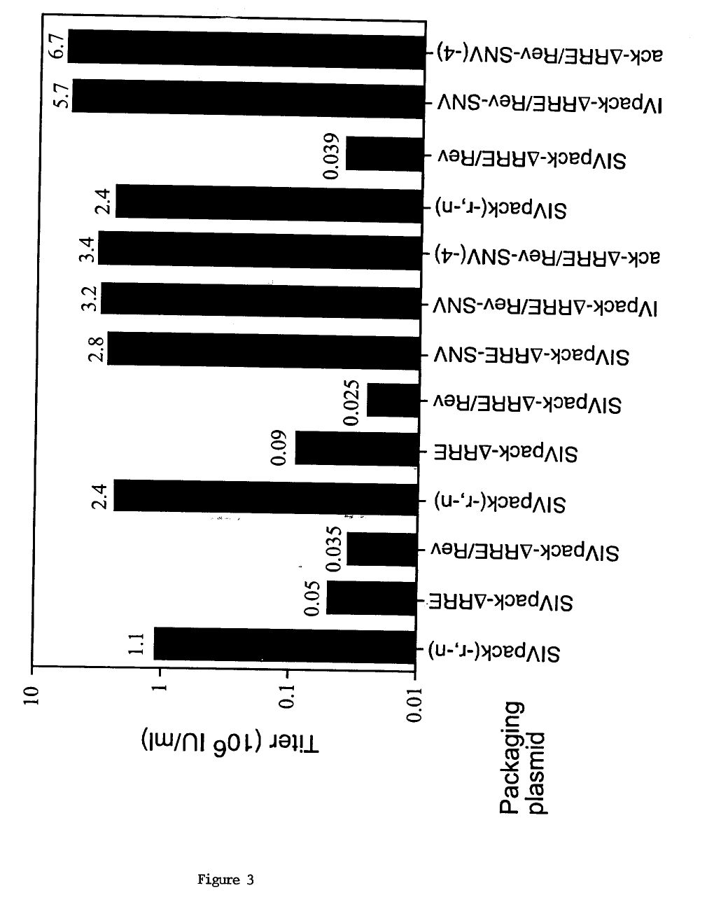

The RRE alone, or both the RRE and rev were deleted from SIVpack (−r,−n), to produce SIVpack-ΔRRE and SIVpack-ΔRRE/Rev (FIG. 1, panel C), and vector production was performed by co-transfection with a transfer vector (HIV-GFP or DSIV-1), and HCMV-VSVG (FIG. 3). Removal of the RRE decreased vector titers by about 22-fold when using HIV-GFP (0.05×10⁶ IU/ml), and by 27-fold when using DSIV-1 (0.09×10⁶ IU/ml) when compared to the RRE/Rev-containing. SIVpack(−r,−n) (1.1×10⁶ IU/ml). Removal of both the RRE and Rev from the packaging construct also produced significant decreases in vector titers (about 31-fold for HIV-GFP [0.035×10⁶ IU/ml] and 96-fold for DSIV-1 [0.025×10⁶ lU/ml]; FIG. 3).

To examine whether optimal vector production would be influenced by the presence of the RRE in the transfer plasmid, a mutant was derived, in which the RRE was deleted from DSIV-1 (DSIV-1ΔRRE; FIG. 1, panel B). We expected that absence of the RRE from DSIV-1ΔRRE would impair expression of this transfer vector, and therefore affect the vector titer. This, however, did not appear to be the case, because co-transfection of DSIV-1ΔRRE with SIVpack(−r, −n) produced no change in the vector titer. Thus, removal of the RRE from the SIV transfer vector appears to have no effect on vector titers and, therefore, we conclude that expression of DSIV-1ΔRRE is RRE/Rev-independent.

Substitution of Rev and the RRE With the SNV 5′ LTR

Various heterologous RNA elements, such as the constitutive transport element (CTE) have been shown to replace the essential requirement for Rev/RRE in HIV-1 and -2 based packaging systems (Zolotukhin, Valentin et al. 1994; Srinivasakumar, Chazal et al. 1997; Corbeau, Kraus et al. 1998; Mautino, Keiser et al. 2000). Recently, Butsch et al. observed that the 5′ terminus of the spleen necrosis virus (SNV) RNA contains a novel post-transcriptional control element (PCE) that facilitates Rev/RRE-independent expression of HIV-1 Gag reporter plasmids (Butsch, Hull et al. 1999).

The SNV PCE was substituted for the RRE and rev function in our SIVmac packaging plasmid-the SV40 promoter of SIVpack was replaced by SNV 5′-long terminal repeat (LTR), which contains the PCE (FIG. 1, panel C). Substitution of the SNV 5′ LTR in SIVpack-ΔRRE (SIVpack-ΔRRE-SNV+DSIV-1) produced a titer of 2.8×10⁶ IU/ml, a 31-fold increase when compared to 0.09×10⁶ IU/ml obtained with to SIVpack-ΔRRE+DSIV-1. In addition, inclusion of the SNV 5′ LTR in SIVpack-ΔRRE/Rev (SIVpack-ΔRRE/Rev-SNV+DSIV-1) produced a titer of 3.2×10⁶ IU/ml, a 125-fold increase when compared to 0.025×10⁶ IU/ml obtained with SIVpack-ΔRRE/Rev+DSIV-1. Thus, inclusion of the SNV 5′ LTR in SIVpack was able to compensate for the lack of RRE/Rev and fully sustain vector titers.

SIVpack-ΔRRE/Rev-SNV was initially constructed using SIVpack(−r,−n) as the parent plasmid. Thus, SIVpack-ΔRRE/Rev-SNV is still capable of expressing two accessory genes, vif and vpx. In an effort to generate a RRE/Rev-independent packaging plasmid that lacks all accessory genes, we created SIVpack-ΔRRE/Rev-SNV(−4) by removing vif and vpx from SIVpack-ΔRRE/Rev-SNV. SIVpack-ΔRRE/Rev-SNV(−4)+DSIV-1 yielded a similar titer to its counterpart SIVpack-ΔRRE/Rev-SNV+DSIV-1 (FIG. 3).

Because the minimal transfer vector, DSIV-1ΔRRE, is RRE/Rev independent, we predicted that it would be functional when co-transfected with SIVpack-ΔRRE/Rev-SNV or SIVpack-ΔRRE/Rev-SNV(−4). Co-transfection of DSIV-1 ΔRRE with either of the SNV PCE-containing plasmids yielded vector titers of 5.7×10⁶ IU/ml and 6.2×10⁶ IU/ml, respectively (FIG. 3), which demonstrated the functionality of RRE/Rev-independent vectors.

TABLE 1
Effect of accessory gene deletion in SIVpack.

| Titer (IU/ml) | Titer (IU/ml) |
|---|---|
| Transfer vector: | Transfer vector: |
| HIV-GFP | DSIV-1 |

| Packaging | | Aphidicolin- | | Aphidicolin- |
|---|---|---|---|---|
| Plasmid | Dividing | treated | Dividing | treated |
| SIVpack (−r, −n) | 1.41 × 10⁶ | 0.99 × 10⁶ | 2.5 × 10⁶ | 2.4 × 10⁶ |
| SIVpack (−x, −n) | 1.81 × 10⁶ | 1.0 × 10⁶ | 1.4 × 10⁶ | 1.4 × 10⁶ |
| SIVpack | 1.6 × 10⁶ | 1.77 × 10⁶ | 1.82 × 10⁶ | 1.74 × 10⁶ |
| (−x, −r, −n) | | | | |
| SIVpack (−n) | 1.22 × 10⁶ | 1.24 × 10⁶ | 2.9 × 10⁶ | 2.8 × 10⁶ |
| SIVpack | 1.69 × 10⁶ | 1.51 × 10⁶ | 1.09 × 10⁶ | 0.76 × 10⁶ |
| (−v, −x, −r) | | | | |
| SIVpack (−4) | 1.51 × 10⁶ | 1.62 × 10⁶ | 1.12 × 10⁶ | 0.79 × 10⁶ |
| SIVpack (+4) | 1.26 × 10⁶ | 0.96 × 10⁶ | 2.65 × 10⁶ | 1.53 × 10⁶ |

CEM (10⁶) untreated or treated with aphidicolin were infected with vectors at a dilution of 1:10 in tissue culture medium. At 48 hours post-infection, GFP positive cells were quantitated by flow cytometry. Vector titers were calculated and expressed in infectious units (IU). One infectious unit corresponds to one GFP-positive cell. These results are representative of three independent experiments.

### REFERENCES

Akkina, R. K., Walton, R. M., Chen, M. L., Li, Q. X. Planelles, V. and Chen, I. S. (1996). J Virol 70, 2581-2585.

Amado, R. G. and Chen, I. S. (1999). Science 285, 674-676.

Arya, S. K. and Gallo, R. C. (1996). Proc Natl Acad Sci USA 93, 4486-4491.

Arya, S. K., Zamani, M. and Kundra, P. (1998). Hum Gene Ther 9, 1371-1380.

Bartz, S. R. and Emerman, M. (1999). J Virol 73, 1956-1963.

Boris-Lawrie, K. and Temin, H. M. (1995). J Virol 69, 1920-1924.

Bray, M., Prasad, S., Dubay, J. W., Hunter, E., Jeang, K. T., Rekosh, D. and Hammarskjold, M. L. (1994). Proc Natl Acad Sci USA 91, 1256-1260.

Burns, J. C., Friedmann, T., Driever, W., Burrascano, M. and Yee, J. K. (1993). Proc Natl Acad Sci USA 90, 8033-8037.

Butsch, M., Hull, S., Wang, Y., Roberts, T. M. and Boris-Lawrie, K. (1999). J Virol 73, 4847-4855.

Campbell, B. J. and Hirsch, V. M. (1997). J Virol 71, 5593-5602.

Chinnasamy, D., Chinnasamy, N., Enriquez, M. J., Otsu, M., Morgan, R. A. and Candotti, F. (2000). Blood 96, 1309-1316.

Cochrane, A. W., Jones, K. S., Beidas, S., Dillon, P. J., Skalka, A. M. and Rosen, C. A. (1991). J Virol 65, 5305-5313.

Connor, R. I., Chen, B. K., Choe, S. and Landau, N. R. (1995). Virology 206, 935-944.

Corbeau, P., Kraus, G. and Wong-Staal, F. (1998). Gene Ther 5, 99-104.

Desrosiers, R. C., Lifson, J. D., Gibbs, J. S., Czajak, S. C., Howe, A. Y., Arthur, L. O. and Johnson, R. P. (1998). J Virol 72, 1431-1437.

Embretson, J. E. and Temin, H. M. (1987). J Virol 61, 2675-2683.

Fletcher, T. M., Brichacek, B., Sharova, N., Newman, M. A., Stivahtis, G., Sharp, P. M., Emerman, M., Hahn, B. H. and Stevenson, M. (1996). Embo J 15, 6155-6165.

Follenzi, A., Ailles, L. E., Bakovic, S., Geuna, M. and Naldini, L. (2000). Nat Genet 25, 217-222.

Frankel, A. D. and Young, J. A. (1998). Annu Rev Biochem 67, 1-25.

Hattori, N., Michaels, F., Fargnoli, K., Marcon, L., Gallo, R. C. and Franchini, G. (1990). Proc Natl Acad Sci USA 87, 8080-8084.

Heinzinger, N. K., Bukinsky, M. I., Haggerty, S. A. Ragland, A. M., Kewalramani, V., Lee, M. A., Gendelman, H. E., Ratner, L., Stevenson, M. and Emerman, M. (1994). Proc Natl Acad Sci USA 91, 7311-7315.

Kafri, T., Blomer, U., Peterson, D. A., Gage, F. H. and Verma, I. M. (1997). Nat Genet 17, 314-317.

Kaye, J. F. and Lever, A. M. (1998). J Virol 72, 5877-5885.

Kestler, H. W., Ringler, D. J., Panicaly, D. L., Sehgal, P. K., Daniel, M. D. and Desrosiers, R. C. (1991). Cell 65, 651-662.

Kim, V. N., Mitrophanous, K., Kingsman, S. M. and Kingsman, A. J. (1998). J Virol 72. 811-816.

Kimpton, J. and Emerman, M. (1992). J Virol 66, 2232-2239.

Kordower, J. H., Emborg, M. E., Bloch, J., Ma, S. Y., Chu, Y., Leventhal, L., McBride, J., Chen, E. Y., Palfi, S., Roitberg, B. Z., Brown, W. D., Holden, J. E., Pyzalski, R., Taylor, M. D., Carvey, P., Ling, Z., Trono, D., Hantraye, P., Deglon, N. and Aebischer, P. (2000). Science 290, 767-773.

Landau, N. R. and Littman, D. R. (1992). J Virol 66, 5110-5113.

Maldarelli, F., Martin, M. A. and Strebel, K. (1991). J Virol 65, 5732-5743.

Malim, M. H., Hauber, J., Le, S. Y., Maizel, J. V. and Cullen, B. R. (1989). The HIV-1 rev trans-activator acts through a structured target sequence to activate nuclear export of unspliced viral mRNA. Nature 338, 254-257.

Mangeot, P. E., Negre, D., Dubois, B., Winter, A. J., Leissner, P., Mehtali, M., Kaiserlian, D., Cosset, F. L. and Darlix, J. L. (2000). J Virol 74, 8307-8315.

Marthas, M. L., Sutjipto, S., Higgins, J., Lohman, B., Torten, J., Luciw, P. A., Marx, P. A. and Pedersen, N. C. (1990). J Virol 64, 3694-3700.

Mautino, M. R., Keiser, N. and Morgan, R. A. (2000). Gene Ther 7, 1421-1424.

Mitrophanous, K., Yoon, S., Rohll, J., Patil, D., Wilkes, F., Kim, V., Kingsman, S., Kingsman, A. and Mazarakis, N. (1999). Gene Ther 6, 1808-1818.

Mochizuki, H., Schwartz, J. P., Tanaka, K., Brady, R. O. and Reiser, J. (1998). J Virol 72, 8873-8883.

Naldini, L., Blomer, U., Gallay, P., Ory, D., Mulligan, R., Gage, F. H., Verma, I. M. and Trono, D. (1996). Science 272, 263-267.

Nasioulas, G., Zolotukhin, A. S., Tabernero, C., Solomin, L., Cunningham, C. P., Paviakis, G. N. and Felber, B. K. (1994). J Virol 68, 2986-2993.

Parolin, C., Dorfman, T., Palu, G., Gottlinger, H. and Sodroski, J. (1994). J Virol 68. 3888-3895.

Pasquinelli, A. E., Ernst, R. K., Lund, E., Grimm, C., Zapp, M. L., Rekosh, D., Hammarskjold, M. L. and Dahlberg, J. E. (1997). Embo J 16, 7500-7510.

Planelles, V., Jowett, J. B. M., Li, Q. X., Xie, Y., Hahn, B. and Chen, I. S. Y. (1996). J Virol 70, 2516-2524.

Poeschla, E., Corbeau, P. and Wong-Staal, F. (1996). Proc Natl Acad Sci USA 93. 11395-11399.

Poeschla, E. M., Wong-Staal, F. and Looney, D. J. (1998). Nat Med 4, 354-357.

Purvis, S. F., Jacobberger, J. W., Sramkoski, R. M., Patki, A. H. and Lederman, M. M. (1995). AIDS Res Hum Retroviruses 11, 443-450.

Regier, D. A. and Desrosiers, R. C. (1990). AIDS Res Hum Retroviruses 6, 1221-1231.

Rizvi, T. A. and Panganiban, A. T. (1992). J Med Primatol 21, 69-73.

Rizvi, T. A. and Panganiban, A. T. (1993). J Virol 67, 2681-2688.

Rizvi, T. A., Schmidt, R. D., Lew, K. A. and Keeling, M. E. (1996). Virology 222.457-463.

Roberts, T. M. and Boris-Lawrie, K. (2000). J Virol 74, 8111-8118.

Schnell, T., Foley, P., Wirth, M., Munch, J. and Uberla, K. (2000). Hum Gene Ther 11, 439-447.

Schwartz, S., Campbell, M., Nasioulas, G., Harrison, J., Felber, B. K. and Pavlakis, G. N. (1992). J Virol 66, 7176-7182.

Schwartz, S., Felber, B. K. and Pavlakis, G. N. (1992). J Virol 66, 150-159.

Srinivasakumar, N., Chazal, N., Helga-Maria, C., Prasad, S., Hammarskjold, M. L. and Rekosh, D. (1997). J Virol 71, 5841-5848.

Stefano, K. A., Collman, R., Kolson, D., Hoxie, J., Nathanson, N. and Gonzalez-Scarano, F. (1993). J Virol 67, 6707-6715.

Tristem, M., Marshall, C., Karpas, A. and Hill, F. (1992). Embo J 11, 3405-3412.

Uchida, N., Sutton, R. E., Friera, A. M., He, D., Reitsma, M. J., Chang, W. C., Veres, G., Scollay, R. and Weissman, I. L. (1998). Proc Natl Acad Sci USA 95, 11939-11944.

Unger, R. E., Marthas, M. L., Pratt-Lowe, E., Padrid, P. A. and Luciw, P. A. (1992). J Virol 66, 5432-5442.

White, S. M., Renda, M., Nam, N. Y., Klimatcheva, E., Zhu, Y., Fisk, J., Halterman, M., Rimel, B. J., Federoff, H., Pandya, S., Rosenblatt, J. D. and Planelles, V. (1999). J Virol 73, 2832-2840.

Zennou, V., Petit, C., Guetard, D., Nerhbass, U., Montagnier, L. and Charneau, P. (2000). Cell 101, 173-185.

Zolotukhin, A. S., Valentin, A., Paviakis, G. N. and Felber, B. K. (1994). J Virol 68, 7944-7952.

Zufferey, R., Donello, J. E., Trono, D. and Hope, T. J. (1999). J Virol 73, 2886-2892.

Zufferey, R., Dull, T., Mandel, R. J., Bukovsky, A., Quiroz, D., Naldini, L. and Trono, D. (1998). J Virol 72, 9873-9880.

Zufferey, R., Nagy, D., Mandel, R. J., Naldini, L. and Trono, D. (1997). Nat Biotechnol 15, 871-875.

All references cited herein are hereby incorporated by reference.

What is claimed:

1. A Simian Immunodeficiency Virus (SIV)-derived vector system for transferring a nucleic acid encoding a target molecule to a host cell, comprising:
(a) a transfer vector containing a nucleic acid sequence encoding a target molecule wherein the nucleic acid sequence is operably linked to a promoter and a SIV packaging sequence including the portion of the SIV long terminal repeat (LTR) sequences necessary to package the SIV RNA into the SIV virion;
(b) a packaging vector derived from an SIV strain and which has at least one accessory gene deleted, which further contains a SIV gag gene encoding a gag protein, wherein the gag gene is operably linked to a promoter and a polyadenylation sequence;
(c) an env vector containing an env gene encoding a functional envelope protein from a virus other than a lentivirus, wherein the env gene is operably linked to a promoter and a polyadenylation sequence; and
(d) a SIV pol gene encoding a pol protein on one of the first two vectors or on at least a third vector, wherein said lentiviral pol gene is operably linked to a promoter and a polyadenylation sequence;
wherein only said transfer vector contains said SIV packaging segment to effectively package lentiviral RNA; and wherein the SIV proteins and the envelope protein when expressed in combination form a SIV virion containing an envelope protein around a SIV capsid.

2. The vector system of claim 1, wherein the packaging vector has at least one of the SIV accessory genes vif, vpr, vpx and nef deleted.

3. The vector system of claims 1 or 2, wherein the SIV RRE has been deleted.

4. The vector system of claim 3, wherein a transporter element is used in place of RRE.

5. The vector system of claim 4, wherein the transporter element is a post-transcriptional control element in spleen necrosis vector LTR present in the packaging virus at the 5′ end.

6. The vector system of claim 1, wherein the env gene is vesicular stomatitic virus-G protein.

7. The vector system of claim 1, wherein the target molecule is operably linked to an inducible promoter.

8. The vector system of claim 1, wherein the target molecule is an antisense molecule, a ribozyme, an antibody, a receptor, a cytokine, an angiogenesis modulation or a growth hormone.

9. The vector system of claim 8, wherein the target molecule is a ribozyme directed to a human immunodeficiency virus.

10. The vector system of claim 8, wherein the ribozyme or antisense molecule is capable of transplicing.

11. The vector system of claim 1, wherein the env gene encodes an envelope protein that targets an endocytic compartment.

12. A host cell transfected by the vector system of claim 1.
